(19) United States
(12) Patent Application Publication
(10) Pub. No.: US 2023/0192872 A1
(43) Pub. Date: Jun. 22, 2023

(54) TOCILIZUMAB FOR THE TREATMENT OF VIRAL INFECTIONS

(71) Applicant: The University of Chicago, Chicago, IL (US)

(72) Inventors: Mark J. RATAIN, Chicago, IL (US); Garth W. STROHBEHN, Chicago, IL (US); Pankti D. REID, Chicago, IL (US); Brian L. HEISS, Chicago, IL (US)

(21) Appl. No.: 17/995,225

(22) PCT Filed: Apr. 1, 2021
(86) PCT No.: PCT/US2021/025444

§ 371 Date: Sep. 30, 2022

Related U.S. Application Data
(60) Provisional application No. 63/003,729, filed on Apr. 1, 2020.
(60) Provisional application No. 63/018,956, filed on May 1, 2020.

(51) Int. Cl. C07K 16/28; A61K 45/06; A61K 31/706; G01N 33/68; A61P 31/14

(52) U.S. Cl. CPC C07K 16/2866; A61K 45/06; A61K 31/706; G01N 33/6869; A61P 31/14; A61K 2039/545

(57) ABSTRACT

Provided herein are methods for the treatment of viral infections and inflammation secondary to viral infections (e.g., coronavirus infections (e.g., severe acute respiratory syndrome coronavirus 2 (SARS-CoV-2), etc.) and diseases (e.g., Coronavirus disease 2019 (COVID-19)) associated therewith by the administration of low doses of tocilizumab to a subject. In particular embodiments, provided herein are pharmaceutical compositions comprising doses of 200 mg or less of tocilizumab and methods for the treatment of SARS-CoV-2 infections, COVID-19, inflammation secondary to such infections, and symptoms or other conditions arising as a result thereof.

### FIELD

Provided herein are methods for the treatment of viral infections and inflammation secondary to viral infections (e.g., coronavirus infections (e.g., severe acute respiratory syndrome coronavirus 2 (SARS-CoV-2), etc.), etc.) and diseases (e.g., Coronavirus disease 2019 (COVID-19)) associated therewith by the administration of low doses of tocilizumab to a subject. In particular embodiments, provided herein are pharmaceutical compositions comprising doses of 200 mg or less of tocilizumab and methods for the treatment of SARS-CoV-2 infections, COVID-19, inflammation secondary to such infections, and symptoms or other conditions arising as a result thereof.

### BACKGROUND

Coronavirus disease-2019 (COVID-19), secondary to the severe acute respiratory syndrome coronavirus-2 (SARS-CoV-2), represents an emergent threat to public health, with a quoted mortality rate among inpatients greater than 25% (Ref 1; incorporated by reference in its entirety). Containment strategies have been employed to varying degrees and effects in Western nations, now necessitating mitigation strategies in overwhelmed hospitals to effectively address COVID-19. Limited intensive care unit—(ICU) level resources such as mechanical ventilation and other in-hospital resources such as bedding space have played a significant role in the poor clinical outcomes associated with COVID-19 (2-4). Moreover, the throughput and length of hospitalization of patients requiring acute care beds may play a role in the “logjam” of available resources in a pandemic. Therefore, early identification of patients at risk for clinical decompensation, imminently preventing their decompensation so as to preserve as many ICU-level resources as possible, and decreasing the length of stay of non-critically ill patients are all crucial at a public health level to navigate the COVID-19 pandemic.

### SUMMARY

Provided herein are methods for the treatment of viral infections and inflammation secondary to viral infections (e.g., coronavirus infections (e.g., severe acute respiratory syndrome coronavirus 2 (SARS-CoV-2), etc.), etc.) and diseases (e.g., Coronavirus disease 2019 (COVID-19)) associated therewith by the administration of tocilizumab to a subject. In particular embodiments, provided herein are pharmaceutical compositions comprising doses of 200 mg or less of tocilizumab and methods for the treatment of severe inflammation secondary to SARS-CoV-2 infections, COVID-19, and symptoms or other conditions arising as a result thereof.

In some embodiments, provided herein are methods of treating a subject infected with severe acute respiratory syndrome coronavirus 2 (SARS-CoV-2) comprising administering an effective dose of tocilizumab (or a biosimilar thereof) to the subject. In some embodiments, the subject suffers from Coronavirus disease 2019 (COVID-19). In some embodiments, the subject suffers from moderate or severe COVID-19. In some embodiments, the subject exhibits symptoms and/or biomarkers of respiratory inflammation. In some embodiments, the respiratory inflammation is aberrant lung inflammation. In some embodiments, the subject suffers from or is exhibiting a systemic inflammatory response. In some embodiments, the subject suffers from or exhibits systemic hyperinflammation (e.g., hyperinflammation manifesting in respiratory and/or pulmonary inflammation, etc.). In some embodiments, the respiratory inflammation is cytokine release syndrome.

In some embodiments, provided herein are methods of treating cytokine release syndrome (CRS) secondary to SARS-CoV-2 infection, comprising administering an effective dose of tocilizumab (or a biosimilar thereof) to a subject suffering from CRS. In some embodiments, provided herein are methods of preventing CRS comprising administering an effective dose of tocilizumab to a subject at risk of developing CRS (e.g., infected with SARS-CoV-2, suffering from COVID-19 (e.g., mild, moderate, or severe), etc.). In some embodiments, an effective dose of tocilizumab to a patient with symptoms (and disease progression) that do not require intensive care.

In some embodiments, provided herein are methods of treating aberrant inflammatory responses arising from a viral infection, the method comprising administering an effective dose of tocilizumab (or a biosimilar thereof) to a subject experiencing symptoms of aberrant inflammation and or displaying biomarkers of aberrant inflammation. In some embodiments, the viral infection is an upper respiratory infection. In some embodiments, the viral infection is a coronavirus infection. In some embodiments, the subject is infected with severe acute respiratory syndrome coronavirus 2 (SARS-CoV-2). In some embodiments, the subject suffers from Coronavirus disease 2019 (COVID-19). In some embodiments, the subject suffers from moderate or severe COVID-19.

In some embodiments, an effective dose comprises 200 mg or less of tocilizumab (e.g., 10 mg, 20 mg, 30, mg, 40 mg, 50 mg, 60 mg, 70 mg, 80 mg, 90 mg, 100 mg, 110 mg, 120 mg, 130, mg, 140 mg, 150 mg, 160 mg, 170 mg, 180 mg, 190 mg, 200 mg, or ranges therebetween). In some embodiments, an effective dose comprises less than 100 mg of tocilizumab. In some embodiments, an effective dose comprises less than 80 mg of tocilizumab. In some embodiments, an effective dose comprises less than 40 mg of tocilizumab. In some embodiments, an effective dose comprises less than 20 mg of tocilizumab. In some embodiments, tocilizumab is administered daily. In some embodiments, tocilizumab is administered two or more times daily (e.g., 2, 3, 4, or more times). In some embodiments, tocilizumab is administered on alternate days or every third or fourth day. In some embodiments, tocilizumab is administered subcutaneously, intravenously, or by any other suitable method.

In some embodiments, a subject is administered multiple doses (e.g., over the course of 2 days, 3 days, 4 days, 5 days, 6 days, 1 week, 2 weeks, or more, or ranges therebetween). In some embodiments, each dose administered is 200 mg or less of tocilizumab (e.g., 10 mg, 20 mg, 30, mg, 40 mg, 50 mg, 60 mg, 70 mg, 80 mg, 90 mg, 100 mg, 110 mg, 120 mg, 130, mg, 140 mg, 150 mg, 160 mg, 170 mg, 180 mg, 190 mg, 200 mg, or ranges therebetween). In some embodiments, the total of all individual doses (e.g., over the course of two weeks or less (e.g., 1 week, 6 days, 5 days, 4 days, 3 days, 2 days, 1 day) is 1000 mg or less (e.g., <900 mg, <800 mg, <700 mg, <600 mg, <500 mg, <400 mg). In some embodiments, the total of all individual doses is 400 mg or less. In some embodiments, the total of all individual doses is 300 mg or less. In some embodiments, the total of all individual doses is 200 mg or less. In some embodiments, the total of all individual doses is 100 mg or less.

In some embodiments, tocilizumab (or a biosimilar thereof) is co-administered with one or more additional therapeutics (e.g., for the treatment of COVID-19, a viral infection, or CRS). In some embodiments, the subject suffers from COVID-19 and the tocilizumab is co-administered with one or more antiviral agents, immunosuppressants, or anti-inflammatory agents. In some embodiments, the subject suffers from COVID-19 and the tocilizumab is co-administered with one or more of lopinavir and ritonavir, umifenovir, hydroxychloroquine, remdesivir, favipiravir, and glucocorticoids.

In some embodiments, tocilizumab (or a biosimilar thereof) is co-administered with the standard of care for the subject and the severity of the COVID-19 symptoms being experienced by the subject. In some embodiments, the subject is hospitalized. In some embodiments, the subject is not mechanically-ventilated. In some embodiments, the subject is an adult. In some embodiments, the subject suffers from COVID-19 pneumonitis.

In some embodiments, provided herein are methods comprising: (a) testing a subject or a biological sample from a subject to determine the appropriateness of tocilizumab administration to treat/prevent cytokine release syndrome as a result of COVID-19; (b) administering a dose of tocilizumab consistent with the methods herein. In some embodiments, testing comprises determining the subject's C-reactive protein level, interleukin-6 level, RT-qPCR testing to determine SARS-CoV-2 infection, chest x-ray, metabolite testing, or any other assays or biomarkers described herein. In some embodiments, methods further comprise (c) testing a subject or a biological sample from a subject to determine the effectiveness of the tocilizumab administration. In some embodiments, methods further comprise (d) determining a treatment course of action based on the testing of step (a) and/or step (c).

### BRIEF DESCRIPTION OF THE DRAWINGS

FIG. 1. Criteria for critical COVID-19.

FIG. 2. Group A. Clinical, laboratory, and epidemiologic characteristics of hospitalized, non-critically ill patients with COVID-19 pneumonitis at higher risk for clinical decompensation, ICU utilization, and COVID-19 disease-related mortality.

FIG. 3. Group B. Clinical and laboratory characteristics of hospitalized, non-critically ill patients with COVID-19 pneumonitis at relatively lower risk for clinical decompensation, ICU utilization, and COVID-19 disease-related mortality.

FIG. 4. Utilization of low-dose tocilizumab in hospitalized, non-critically ill patients with COVID-19 disease to prevent clinical decompensation, ICU utilization, and COVID-19-related death.

FIG. 5. Patient flow chart. Thirty-two eligible patients consented to participate in the trial, of which 12 were assigned to Group A and 20 were assigned to Group B on the bases of magnitude of C-reactive protein elevation and epidemiologic risk factors for COVID-19 related mortality. Group A patients received either 200 or 120 mg of tocilizumab and were followed for 28 days following drug administration. Group B patients received either 80 or 40 mg of tocilizumab and were followed for 28 days following drug administration. All 32 patients were evaluable for the purposes of primary clinical outcome and 29 were evaluable for biochemical outcome. No patients were lost to follow-up.

FIGS. 6A-F. Temperature and C-reactive protein decrease rapidly following administration of low-dose tocilizumab. (A) Temperature values for patients following eligibility (mean±standard error of mean). (B) Percentage of total (dark gray), Group A (light gray), and Group B (white) patients meeting the primary outcome of Tₘₐₓ₂₄ₕᵣₛ<38.0° C. (C) Relationship between tocilizumab dose and probability of achieving fever resolution. Percentage of patients at the noted tocilizumab dose levels achieving the outcome of Tₘₐₓ₂₄ₕᵣₛ<38.0° C. Bars denote one-sided Fisher's exact test for difference in proportions. P values are as shown in the panel. (D) Representative C-reactive protein values for patients following eligibility (mean±standard error of mean). (E) Percentage of total (dark gray), Group A (light gray), and Group B (white) patients meeting the primary biochemical outcome of CRP decline of 25% at 24 hours. (F) Relationship between tocilizumab dose and probability of achieving CRP decline of 25%. Percentage of patients at the noted tocilizumab dose levels achieving CRP decline of 25%. Bars denote one-sided Fisher's exact test for difference in proportions. P values are as shown in the panel.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “an IL-6 receptor antagonist” is a reference to one or more IL-6 receptor antagonists and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “IL-6 receptor antagonist” refers to an agent (e.g., small molecule, peptide, antibody, antibody fragment, etc.) that binds to IL-6 receptor and inhibits IL-6 receptor biological activity. In some embodiments, an IL-6 receptor antagonist is a competitive antagonist and binds to IL-6 receptor in the same location as a natural IL-6 receptor ligand (e.g., interleukin 6, ciliary neurotrophic factor) and inhibits binding of the natural ligand to the receptor. In some embodiments, an IL-6 receptor antagonist is a non-competitive antagonist and binds to IL-6 receptor in a distinct location from a natural IL-6 receptor ligand but still inhibits the biological activity of IL-6 receptor.

As used herein, the term “IL-6 receptor inhibitor” refers to an agent (e.g., small molecule, peptide, antibody, antibody fragment, aptamer, nucleic acid, etc.) that reduces IL-6 receptor activity or expression. An IL-6 receptor inhibitor may function by any suitable mechanism, including but not limited to reducing/inhibiting expression of IL-6 receptor (e.g., RNAi, antisense RNA, etc.), sequestering IL-6 receptor (e.g., antibody), preventing interaction of IL-6 receptor with other components involved in its function (e.g. IL-6), etc.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the terms “subject at risk for a disease,” or “subject at risk for a condition,” for example, “a subject at risk for cytokine release syndrome refers to a subject with one or more risk factors for developing the disease/condition (e.g., SARS-CoV-2 infection, suffering form COVID-19, etc.). Depending upon the specific disease, risk factors may include, but are not limited to, gender, age, genetic predisposition, environmental exposures, infections, and previous incidents of diseases, lifestyle, etc.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be by parenteral administration (e.g., intravenously, subcutaneously, etc.).

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “co-administration” and “co-administering” refer to the administration of at least two agent(s) (e.g., tocilizumab and one or more additional therapeutics) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “instructions for administering,” and grammatical equivalents thereof, includes instructions for using the compositions contained in a kit for the treatment of conditions (e.g., providing dosing, route of administration, decision trees for treating physicians for correlating patient-specific characteristics with therapeutic courses of action).

As used herein, the term “antibody” refers to a whole antibody molecule or a fragment thereof (e.g., fragments such as Fab, Fab′, and F(ab′)₂), it may be a polyclonal or monoclonal antibody, a chimeric antibody, a humanized antibody, a human antibody, etc.

A native antibody typically has a tetrameric structure. A tetramer typically comprises two identical pairs of polypeptide chains, each pair having one light chain (in certain embodiments, about 25 kDa) and one heavy chain (in certain embodiments, about 50-70 kDa). In a native antibody, a heavy chain comprises a variable region, VH, and three constant regions, C_{H1}, C_{H2}, and C_{H3}. The VH domain is at the amino-terminus of the heavy chain, and the C_{H3} domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen-binding site. H3, for example, in certain instances, can be as short as two amino acid residues or greater than 26. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); Chothia, C., and Lesk, A. M. (1987) J. Mol. Biol. 196:901-917; or Chothia, C. et al. Nature 342:878-883 (1989). In the present application, the term “CDR” refers to a CDR from either the light or heavy chain, unless otherwise specified.

As used herein, the terms “anti-IL-6 receptor antibody” or “IL-6 receptor antibody” refer to an antibody which specifically recognizes an antigen and/or epitope presented by the IL-6 receptor.

As used herein, the term “monoclonal antibody” refers to an antibody which is a member of a substantially homogeneous population of antibodies that specifically bind to the same epitope. In certain embodiments, a monoclonal antibody is secreted by a hybridoma. In certain such embodiments, a hybridoma is produced according to certain methods known to those skilled in the art. See, e.g., Kohler and Milstein (1975) Nature 256: 495-499; herein incorporated by reference in its entirety. In certain embodiments, a monoclonal antibody is produced using recombinant DNA methods (see, e.g., U.S. Pat. No. 4,816,567). In certain embodiments, a monoclonal antibody refers to an antibody fragment isolated from a phage display library. See, e.g., Clackson et al. (1991) Nature 352: 624-628; and Marks et al. (1991) J. Mol. Biol. 222: 581-597; herein incorporated by reference in their entireties. The modifying word “monoclonal” indicates properties of antibodies obtained from a substantially-homogeneous population of antibodies, and does not limit a method of producing antibodies to a specific method. For various other monoclonal antibody production techniques, see, e.g., Harlow and Lane (1988) Antibodies: A Laboratory Manual (Cold Spring Harbor Laboratory, Cold Spring Harbor, N.Y.); herein incorporated by reference in its entirety.

As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis.

For example, a “Fab” fragment comprises one light chain and the C_{H1} and variable region of one heavy chain. The heavy chain of a Fab molecule cannot form a disulfide bond with another heavy chain molecule. A “Fab′” fragment comprises one light chain and one heavy chain that comprises additional constant region, extending between the C_{H1} and C_{H2} domains. An interchain disulfide bond can be formed between two heavy chains of a Fab′ fragment to form a “F(ab′)2” molecule.

An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen.

Other antibody fragments will be understood by skilled artisans.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Suitable samples that may find use in embodiments herein include, but are not limited to: blood, plasma, sera, urine, saliva, cells, cell lysates, tissues, tissue homogenates, purified nucleic acids, stool, vaginal secretions, cerebrospinal fluid, allantoic fluid, water, biofilm, soil, dust, food, beverage, agriculture products, plants, etc.

As used herein, the term “biosimilar” refers to a biopharmaceutical which is deemed to be comparable in quality, safety, and efficacy to a reference product marketed by an innovator company (Section 351(i) of the Public Health Service Act (42 U.S.C. 262(i)). Tocilizumab biosimilars may find use in any suitable embodiments herein.

### DETAILED DESCRIPTION

Provided herein are methods for the treatment of viral infections and inflammation secondary to viral infections (e.g., coronavirus infections (e.g., severe acute respiratory syndrome coronavirus 2 (SARS-CoV-2), etc.), etc.) and diseases (e.g., Coronavirus disease 2019 (COVID-19)) associated therewith by the administration of tocilizumab to a subject. In particular embodiments, provided herein are pharmaceutical compositions comprising doses of 200 mg or less of tocilizumab and methods for the treatment of severe inflammation secondary to SARS-CoV-2 infections, COVID-19, and symptoms or other conditions arising as a result thereof.

IL-6 contributes to host defense against infections and tissue injuries. However, exaggerated, excessive synthesis of IL-6 while fighting environmental stress leads to an acute severe systemic inflammatory response known as ‘cytokine storm’, since high levels of IL-6 can activate the coagulation pathway and vascular endothelial cells but inhibit myocardial function. Remarkable beneficial effects of IL-6 blockade therapy using a humanized anti-IL-6 receptor antibody, tocilizumab have been observed in patients with cytokine release syndrome complicated by T-cell engaged therapy (Tanaka et al. Immunotherapy. 2016 July; 8(8):959-70.; incorporated by reference in its entirety).

Accumulating evidence indicates that a subgroup of patients infected with SARS-CoV-2, particularly those suffering from severe COVID-19, exhibit a cytokine release syndrome (e.g., acute cytokine storm). Clinical observations (e.g., fever, confusion) and laboratory features (e.g., hyperferritinemia, lymphopenia, prolonged prothrombin time, elevated lactate dehydrogenase, elevated interleukin (IL) 6, elevated C-reactive protein, elevated soluble CD25, etc.) of critically ill COVID-19 patients infected with COVID-19 indicates the presence of cytokine release syndrome (CRS), in some cases resulting in respiratory distress syndrome and multi-organ failure (Chen et al. Lancet. 2020 Jan. 30; 395(10223):P507—P513.; Lei et al. Chin J Tuberc Respir Dis. 2020 February; 43:E005.; Wang et al. JAMA. 2020 Feb. 7.; incorporated by reference in their entireties). Indeed, many of the diagnostic criteria for CRS are reported present in COVID-19 patients under intensive care.

Tocilizumab is a monoclonal antibody that competitively inhibits the binding of interleukin-6 (IL-6) to its receptor (IL-6R). Inhibiting the entire receptor complex prevents IL-6 signal transduction to inflammatory mediators that summon B and T cells. Tocilizumab was originally developed for outpatient, every four-weeks administration in rheumatoid arthritis (RA), giant cell arteritis, polyarticular juvenile idiopathic arthritis, and systemic juvenile idiopathic arthritis at a labeled dose of 8 mg/kg, with approval based on improvement in the American College of Rheumatology (ACR) 20 score (Ref 17; incorporated by reference in its entirety). Dose-finding studies revealed a dose-related reduction in Disease Activity Score in 28 joints (DAS28) beyond 4 weeks at doses 4 mg/kg and 8 mg/kg. In its subsequent new drug application for treating CRS in chimeric antigen receptor T cell (CAR T) therapy, no formal dose-finding study was performed and tocilizumab was approved after the 8 mg/kg dose, provisioned up to four times at least eight hours apart, led to resolution of CRS within a 14-day period of time (the primary outcome measure for these purposes). Response data from Food and Drug Administration (FDA) filings, however, suggest a 4 mg/kg dose had similar efficacy (Ref 18; incorporated by reference in its entirety). Lower doses were not studied (Ref 18; incorporated by reference in its entirety). No common CRS biomarkers such as ferritin or C-reactive peptide (CRP) response were included as part of tocilizumab's FDA filings.

Provided herein are compositions (e.g., comprising tocilizumab) and methods (e.g., administering tocilizumab or co-administering tocilizumab with additional therapies) for the treatment of SARS-CoV-2 infection, COVID-19, cytokine storm, and symptoms/conditions arising therefrom (e.g., lung damage, organ failure, etc.). In some embodiments, provided herein is the use of unit doses of tocilizumab in the range of 10-200 mg, repeated at intervals (e.g., 24 hours, 48 hours, etc.) as required for up to ten doses (e.g., 2, 3, 4, 5, 6, 7, 8, 9, 10, or ranges therebetween), with a maximum total dose of, for example, 400 mg. In some embodiments, provided herein is the combination of these lower doses of tocilizumab with antiviral drugs, other immunosuppressive therapies, etc. In some embodiments, tocilizumab is administered either intravenously or subcutaneously. In some embodiments, dosing is guided by laboratory and/or clinical studies (e.g., with a particular focus on CRP and IL-6 assays).

Experimental evidence supports the conclusion that some viruses, including SARS-Cov-2, are dependent on IL-6 for their ability to replicate rapidly. Therefore, tocilizumab (and other IL-6-targeted agents) are both antiviral and immunosuppressive. In some embodiments, tocilizumab (and related drugs) are used alone or in addition to other therapies (e.g., that have direct antiviral effects), for example, remdesivir, plasma therapy, etc. In embodiments in which viral replication is stimulated by IL-6, targeting IL-6 early in the disease process is safe, and more effective than delaying such treatment. In some embodiments, low-dose tocilizumab (e.g., sufficient to block IL6 signaling for a week or less) is effective, particularly if administered earlier in the disease course.

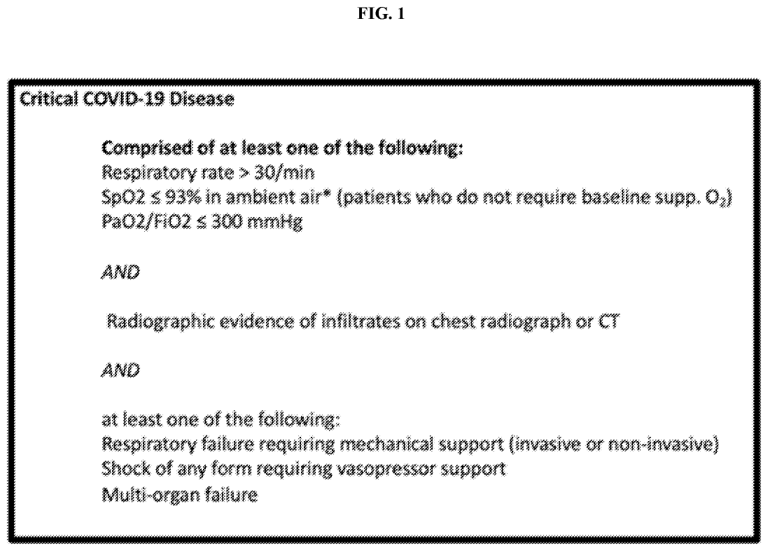

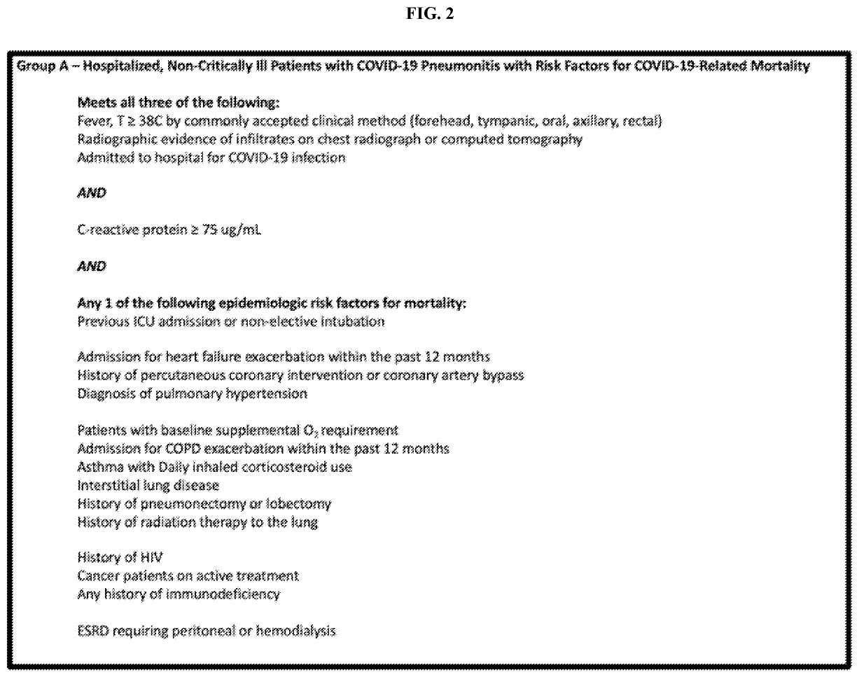

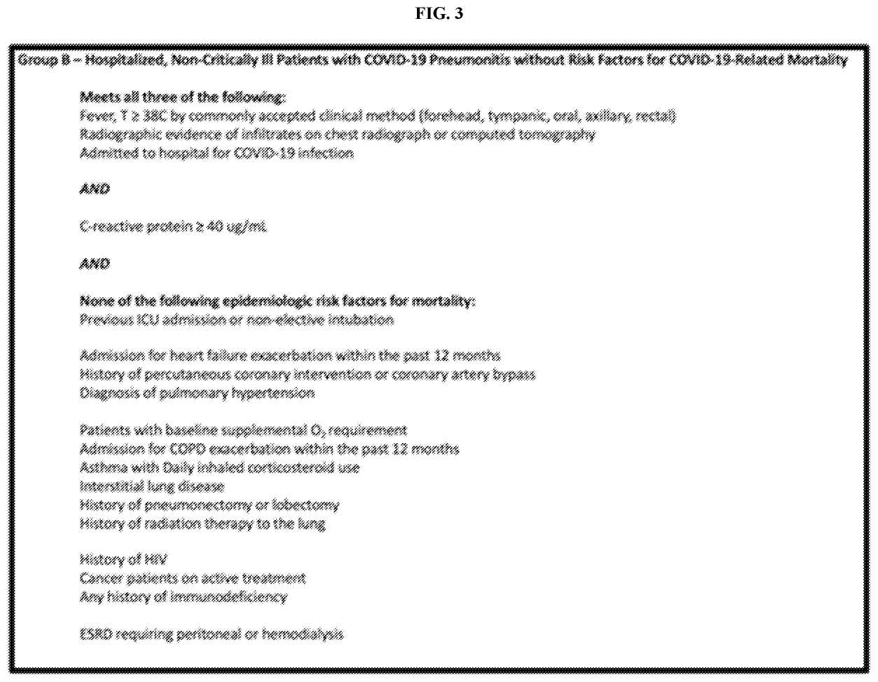

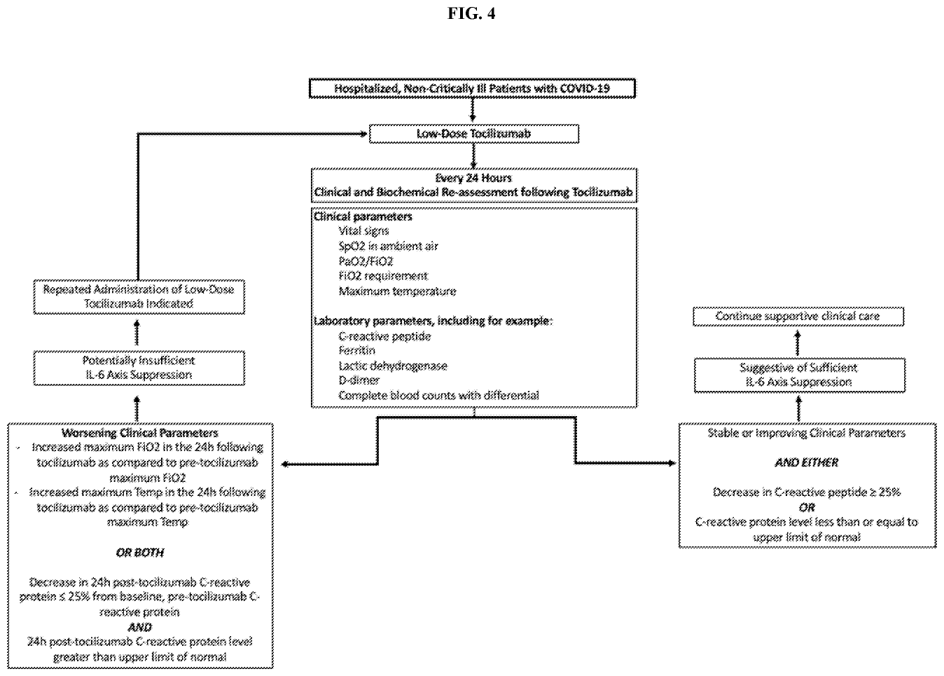

Varying clinical presentations of COVID-19 disease exist. The pneumonitis associated with COVID-19 disease exists on a continuum, ranging from (on one extreme) bilateral infiltrates and associated respiratory failure, hyperinflammation, and septic shock leading to death to (at the other extreme) absence of pulmonary infiltrates and very mild symptoms. Patients meet diagnostic criteria for COVID-19 pneumonitis when they have: 1) positive SARS-CoV-2 viral polymerase chain reaction test and 2) evidence of an infiltrate on chest imaging. Patients with COVID-19 disease may be admitted to the hospital for varying reasons. It is possible that patients with COVID-19 disease may be admitted to the hospital and not have COVID-19 pneumonitis. Those patients with COVID-19 disease who do not have pulmonary infiltrates are generally at low risk for progression to pneumonitis and clinical decompensation, ICU resource utilization, and death. Some embodiments herein are primarily focused on hospitalized patients with signs of COVID19 pneumonitis who are not designated to be critically ill as delineated by aforementioned parameters (See FIG. 1). Patients are broadly risk-stratified into those who meet diagnostic criteria for pneumonitis according to their risk of clinical decompensation, intensive care unit (ICU) utilization, and death. The first division in the risk stratification schema is between patients who are critically ill (FIG. 1) and those who are not. Within the subpopulation who are not critically ill, there are those with relatively high risk for decompensation, ICU utilization, and COVID-19-related mortality (Group A (“high risk”); FIG. 2) and those with relatively low risk for these same outcomes (Group B (“high risk”); FIG. 3).

Critically ill patients with COVID-19 pneumonitis who are already utilizing near-ICU or ICU-level resources (FIG. 1) have polymerase chain reaction (PCR)-proven SARS-CoV-2 infection, radiographic evidence of infiltrates, and physiologic signs of pneumonitis and COVID-19 related hyperinflammation marked by one or more of either respiratory rate greater than or equaled to 30 per minute, oxygen saturation of 93% or less in ambient air (for patients without baseline supplemental oxygen requirement), or a partial pressure of oxygen (PaO2)-fraction of inspired oxygen (FiO2) ratio of less than or equal to 300 mmHg. In addition, critical patients are defined as having one or more of either respiratory failure requiring mechanical ventilation (invasive or non-invasive), shock of any form requiring the use of a vasopressor medication, or multi-organ failure.

Non-critically ill patients that are admitted to the hospital, but do not meet criteria for critical COVID-19 disease still have risk of clinical decompensation, utilization of ICU-level resources, and ultimately COVID-19 disease-related mortality. This population of patients can itself be thought of as two subpopulations: One has relatively high risk for progression to critical COVID-19 disease and ultimately COVID-19 disease-related mortality (FIG. 2) (hereafter ‘Group A’). Risk factors, as determined by a consensus of a multi-disciplinary panel of experts, include epidemiologic risk factors related to medical history and laboratory-based signs of hyperinflammation. The second subpopulation is comprised of patients (hereafter ‘Group B’) (FIG. 3) who have COVID-19 disease but lack the epidemiologic or laboratory-based risk factors known to marginally increase the probability of progression to critical COVID-19 disease or COVID-19-related mortality. Both groups have risk of clinical decompensation, utilization of ICU-level resources, and COVID-19 disease-related mortality and may benefit from early treatment with medications aimed at managing COVID-19 disease-related CRS. As a public health measure, early treatment may help to preserve limited ICU resources.

COVID-19's high mortality is in part be driven by hyperinflammation resembling the cytokine storm often identified in hemophagocytic lymphohistiocytosis and cytokine release syndrome (CRS), also known as cytokine storm syndrome (CSS). In some embodiments, immunosuppressive therapies used to treat CRS—such as the interleukin-6 (IL-6) receptor-targeted monoclonal antibodies tocilizumab or sarilumab and IL-6 targeted monoclonal antibody siltuximab—are employed to reduce mortality in COVID-19. Emerging evidence shows marked elevation of serum IL-6, C-reactive protein (CRP), lactic dehydrogenase, and ferritin in patients with COVID-19, with the degree of elevation correlating with severity of disease (Refs. 1, 6, 7; incorporated by reference in their entireties). Data indicate that IL-6 axis suppression has promise. Retrospective analysis of severe to critical COVID-19 patients receiving tocilizumab 400 mg (approximately 6.67 mg/kg using average Chinese body weight) demonstrated that greater than 75% of patients had rapid resolution (i.e., within 24-72 hours following administration) of both clinical and biochemical signs (fever and CRP, respectively) of hyperinflammation with only a single tocilizumab dose (Ref 6); incorporated by reference in its entirety.

In some embodiments, the compositions and methods herein find use in the treatment of a viral infection and/or diseases, conditions, or symptoms arising therefrom. In some embodiments, tocilizumab is administered (alone or co-administered with other agents) for the treatment of a viral infection and/or diseases, conditions, or symptoms arising therefrom (e.g., CRS).

The viral infection to be treated or prevented can be caused by any virus, including but not limited to, Arbovirus, Adenoviridae, Arenaviridae, Arterivirus, Astroviridae, Baculoviridae, Bimaviridae, Birnaviridae, Bunyaviridae, Caliciviridae, Caulimoviridae, Circoviridae, Coronaviridae, Cystoviridae, Dengue, EBV, HIV, Deltaviridae, Filviridae, Filoviridae, Flaviviridae, Hepadnaviridae (Hepatitis), Herpesviridae (such as, Cytomegalovirus, Herpes Simplex, Herpes Zoster), Iridoviridae, Mononegavirus (e.g., Paramyxoviridae, Morbillivirus, Rhabdoviridae), Myoviridae, Orthomyxoviridae (e.g., Influenza A, Influenza B, and parainfluenza), Papiloma virus, Papovaviridae, Paramyxoviridae, Prions, Parvoviridae, Phycodnaviridae, Picomaviridae (e.g. Rhinovirus, Poliovirus), Poxviridae (such as Smallpox or Vaccinia), Potyviridae, Reoviridae (e.g., Rotavirus), Retroviridae (HTLV-I, HTLV-II, Lentivirus), Rhabdoviridae, Tectiviridae, Togaviridae (e.g., Rubivirus), or any combination thereof. In particular embodiments, the viral infection is caused by a virus of the Coronaviridae family (e.g., a coronavirus). In some embodiments, the viral infection is an upper respiratory infection (e.g., SARS, MEMS, SARS-CoV, SARS-CoV-2, rhinoviruses, adenoviruses, bocavirus, etc.). In another embodiment, the infection and/or a disease or condition resulting therefrom is capable of producing or resulting in a cytokine release syndrome (e.g., acute cytokine storm).

In some embodiments, a subject that is infected with SARS-CoV-2 (e.g., tested positive for SARS-CoV-2) is treated with tocilizumab to prevent/treat symptoms and complications resulting therefrom, such as a cytokine storm and lung damage or organ failure resulting therefrom. In some embodiments, a subject suffers from COVID-19 as a result of a SARS-CoV-2 infection.

COVID-19 may be classified as “mild,” “moderate,” “severe,” and “critical.” A mild case of COVID-19 (which may develop into a more severe case, but might not) typically consists of a low/moderate fever, respiratory symptoms, cough, aches, and pains. An individual patient might exhibit only a subset of these symptoms. Moderate COVID-19 is defined as a subject displaying evidence of pulmonary infiltrates (by chest X-ray or CT scan), but without a need for supplemental oxygen (or more than the patient's baseline oxygen requirement if using home oxygen). Symptoms of being moderately ill with COVID-19 may include coughing, fever above 37.8° C., chills, shortness of breath, dehydration, and increased tiredness. A severe case of COVID-19 will include the preceding symptoms as well as inflammation in the lungs (e.g., leading to cytokine release), pneumonia, low oxygen levels in the blood, trouble breathing, pain or pressure in the chest, confusion, inability to arouse, difficulty eating or drinking, and bluish lips/face. A critical case of COVID-19 may include severe pneumonia, ARDS (acute respiratory distress syndrome), sepsis, and organ failure.

In some embodiments, tocilizumab is administered to a subject suffering from COVID-19. In some embodiments, tocilizumab is administered to a subject suffering from moderate COVID-19. In some embodiments, tocilizumab is administered to a subject suffering from severe COVID-19. In some embodiments, tocilizumab is administered to a subject in which SARS-CoV-2 has infected their lungs or displaying symptoms indicative of viral infection of the lungs. In some embodiments, tocilizumab is administered to a subject displaying signs/symptoms/biomarkers of lung or other respiratory inflammation. In some embodiments, tocilizumab is administered to a subject suffering from and/or displaying signs/symptoms/biomarkers of cytokine storm syndrome (e.g., acute cytokine storm), also known as cytokine release syndrome. In some embodiments, tocilizumab is administered to a subject suffering from and/or displaying signs/symptoms/biomarkers of aberrant IL receptor-dependent inflammation and/or IL-6-receptor-dependent cytokine storm. In some embodiments, tocilizumab is administered to prevent lung damage, respiratory distress, and secondary bacterial pneumonia.

In some embodiments, a subject is critically ill from COVID-19. In some embodiments, a critically ill patient exhibits one or more of ICU utilization, pneumonitis (e.g., physiologic signs of pneumonitis), bilateral infiltrates (e.g., radiographic evidence of infiltrates), respiratory failure, hyperinflammation, and septic shock, increased respiratory rate (e.g., >20, >25, >30, >35, >40 per minute), low oxygen saturation (e.g., <95%, <94%, <93%, <92%, <91%, or <90% in ambient air), respiratory failure requiring mechanical ventilation, shock of any form (e.g., requiring the use of a vasopressor medication), or organ failure (e.g., multiorgan failure).

In some embodiments, a subject suffering from COVID-19 exhibits mild pneumonitis (e.g., no pulmonary infiltrates, mild symptoms, etc.).

In some embodiments, a subject has a positive test for active SARS-CoV-2 infection. In some embodiments, a subject exhibits signs/symptoms of COVID-19, such as fever (e.g., >100.4), cough, aches/pains, chills, shortness of breath, dehydration, increased tiredness.

In some embodiments, a subject is not critically ill, but at moderate to high risk of the disease advancing to decompensation, ICU utilization, and COVID-19 mortality. In some embodiments, a subject is not critically ill, but exhibits symptoms that warrant hospital admission. Hospital admission criteria includes epidemiologic risk factors related to medical history and laboratory-based signs of hyperinflammation. In some embodiments, a subject is considered to be at high risk of disease advancement if C-reactive protein (CRP) levels are greater than or equal to 10 μg/mL, 15 μg/mL, 20 μg/mL, 25 μg/mL, 30 μg/mL, 35 μg/mL, 40 μg/mL, 45 μg/mL, 50 μg/mL, 55 μg/mL, 60 μg/mL, ≥65 μg/mL, ≥70 μg/mL, ≥75 μg/mL, ≥80 μg/mL, ≥85 μg/mL, or ≥90 μg/mL. In some embodiments, a subject is considered to be at high risk of disease advancement if they exhibit one or more of the following characteristics/criteria: previous intubation, prior heart failure, history of percutaneous coronary intervention, history of coronary artery bypass graft (CABG) surgery, diagnosis of pulmonary hypertension, baseline requirement for supplemental oxygen, diagnosis of interstitial lung disease (ILD), prior hospital admission for chronic obstructive pulmonary disease (COPD) exacerbation, asthma (e.g., with use of daily inhaled corticosteroid), history of pneumonectomy or lobectomy, history of radiation therapy to the lung, history of HIV infection. cancer of any stage and receiving active treatment (e.g., excluding hormonal therapy), history of diagnosed immunodeficiency, or end-stage renal disease (e.g., requiring peritoneal or hemodialysis).

In some embodiments, a subject is not critically ill, but suffering from COVID-19, exhibits pneumonitis, and is at low risk of the disease advancing to decompensation, ICU utilization, and COVID-19 mortality. In some embodiments, a subject is considered to be at low risk of disease advancement if C-reactive protein (CRP) levels are less than 60 μg/mL, <65 μg/mL, <70 μg/mL, <75 μg/mL, <80 μg/mL, <85 μg/mL, or <90 μg/mL. In some embodiments, a subject is considered to be at low risk of disease advancement if C-reactive protein (CRP) levels are less than 75 μg/mL.

In some embodiments, a subject suffers from mild COVID-19. In some embodiments, a subject suffers from COVID-19, but does not exhibit signs of pulmonary or respiratory inflammation (or hyperinflammation). In some embodiments, a subject's symptoms or condition does not indicate anti-inflammatory treatment. In some embodiments, a subject is administered tocilizumab as an outpatient (e.g., the subject is not administered tocilizumab as a hospital inpatient). In some embodiments, a subject self-administers (e.g., subcutaneously) tocilizumab. In some embodiments, a subject has a normal or low level C-reactive protein in the blood (e.g., 10 μg/ml, <10 μg/ml, <5 μg/ml, <4 μg/ml, <3 μg/ml, <2 μg/ml, <1 μg/ml). In some embodiments, a subject has an elevated level of C-reactive protein in the blood (e.g., >10 μg/ml, >20 μg/ml, >30 μg/ml, >40 μg/ml, >50 μg/ml, >60 μg/ml, >70 μg/ml, >75 μg/ml, >100 μg/ml, etc.), indicative of inflammation.

Tocilizumab is typically administered (e.g., for treatment of adults with moderate-to-severe active rheumatoid arthritis (RA)) via intravenous (IV) infusion of subcutaneous injection. For IV infusion, a standard dose for the treatment of RA is 4 mg/kg (e.g., over 60 min) every four weeks (q4Weeks), which may be increased to 8 mg/kg q4Weeks based on clinical response. The IV dose of tocilizumab for the treatment of RA is not to exceed 800 mg/dose every four weeks. Typical doses of tocilizumab for the treatment of RA are 400-800 mg every four weeks.

At an individual patient level, utilizing the minimally effective dose of a drug (tocilizumab) can optimize efficacy with minimalization of adverse events. On a more global scale, the logic behind a grand strategy for COVID-19 pandemic mitigation efforts is to proactively utilize one rare resource (tocilizumab) to prevent utilization of another, more limited resource (ICU-level care), rather than waiting until a patient is already committed to the most limited resource (ICU-level care) to then expend another limited resource (tocilizumab). The goal of the overarching treatment strategy is to provision potentially efficacious therapy to the greatest number of patients likely to benefit from tocilizumab while still preserving an adequate supply for critically ill patients (FIG. 4) (Ref 12; incorporated by reference in its entirety).

In some embodiments, provided herein are lower than standard RA doses of tocilizumab for the treatment or prevention of symptoms/conditions arising from viral infection (e.g., coronavirus infection, SARS-CoV-2 infection, etc.) and diseases arising therefrom (e.g., COVID-19), and in particular CRS and other aberrant inflammatory processes that may negatively affect patients.

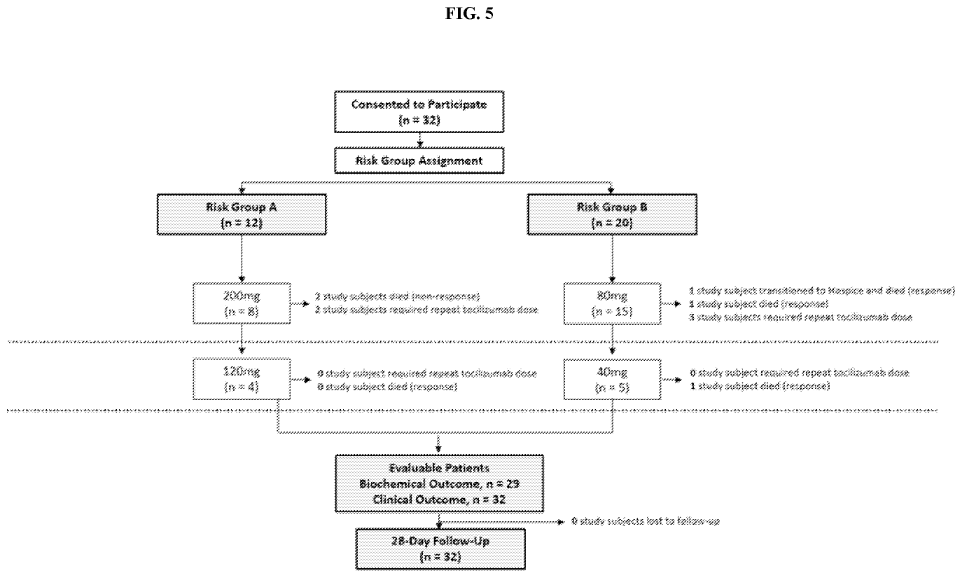

Tocilizumab's effectiveness in CRS after CAR-T therapy has led to off-label treatment of CRS from COVID-19 infections with preliminary evidence showing efficacy (Ref 6; incorporated by reference in its entirety). Unfortunately, tocilizumab is in limited supply. In this context, provided herein are alternative treatment regimens for supportive care of COVID-19 (FIG. 5) with the aim of providing therapy for the greatest number of patients likely to benefit. Hospitalized, non-critically ill patients with COVID-19 with and without risk factors for developing severe disease may be prime candidates for treatment with tocilizumab. Given the much lower IL-6 concentrations in COVID-19 compared to CRS, in some embodiments herein single doses less than 200 mg are provided as effective doses. To date, tocilizumab 400 mg is the adopted standard of care dose, representing a dose of approximately 5 mg/kg (based on average body weight in the United States). The speed with which both clinical and biochemical improvements in COVID-19-related hyperinflammation are seen in patients with COVID-19 treated with tocilizumab suggests rapid readout within 24 hours is possible (FIG. 5). Patients with clinical evidence (e.g., declining SpO₂ in room air, rising PaO₂/FiO₂ ratio, persistent fever, or developing need for vasopressor support or other ICU-level care) or biochemical evidence (e.g., C-reactive peptide decrease of less than 25% in the 24 hours following tocilizumab dose) of insufficient IL-6 axis-suppression can be re-dosed at 24- to 48-hour intervals. Similar approaches may be useful in safely reducing the labeled doses of other IL-6 axis-suppressing therapies and IL-1 antagonists (e.g., anakinra) to more COVID-19-appropriate doses, thereby increasing effective supply.

In some embodiments, tocilizumab is provided in 10-200 mg doses (e.g., 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, and ranges therebetween (e.g., 20-80 mg)). In some embodiments, the dose is adjusted based on the effectiveness and/or side effects of initial dosing. For example, if the initial dose is/appears effective and/or side effects are/appear significant, the dose may be reduced (e.g., 10-100 mg (e.g., 10 mg, 20 mg, 30 mg, 40 mg, 50 mg, 60 mg, 70 mg, 80 mg, 90 mg, 100 mg, and ranges therebetween (e.g., 30-50 mg)). If the initial dose is/appears ineffective and/or side effects are/appear insignificant, the dose may be increased (e.g., 100-200 mg (e.g., 100 mg, 110 mg, 120 mg, 130 mg, 140 mg, 150 mg, 160 mg, 170 mg, 180 mg, 190 mg, 200 mg, and ranges therebetween).

In some embodiments, dosing is guided by clinical and/or laboratory studies, with a particular focus on CRP and IL-6 assays.

In some embodiments, the dose is adjusted based on the effectiveness and/or side effects of initial dosing. For example, if the initial dose is/appears effective and/or side effects are/appear significant, the dose may be reduced (e.g., 20-100 mg (e.g., 20 mg, 30 mg, 40 mg, 50 mg, 60 mg, 70 mg, 80 mg, 90 mg, 100 mg, and ranges therebetween (e.g., 30-50 mg)). If the initial dose is/appears ineffective and/or side effects are/appear insignificant, the dose may be increased (e.g., 200-400 mg (e.g., 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, or ranges therebetween).

In some embodiments, the aforementioned doses are administered daily, or once every 2 days, or 3 days. In some embodiments, doses are administered based on symptoms, biomarkers, and/or clinical evaluation.

In some embodiments, 400 mg or less of tocilizumab is administered daily (q1day) (e.g., 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, or ranges therebetween).

In some embodiments, 400 mg or less of tocilizumab is administered on alternating days (q2 day) (e.g., 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, or ranges therebetween).

In some embodiments, doses are administered q1day, q2day, q3day, q4day, q5 day, q6day, q1week, q2week, q3week, q4week, q5week, q6 week, or any regimens therebetween.

In some embodiments, a first dose (e.g., of less than 400 mg (e.g., 50 mg, 80 mg, 200 mg, ranges therebetween, etc.) of tocilizumab is administered. In some embodiments, a second dose (e.g., of less than 400 mg (e.g., 50 mg, 80 mg, 200 mg, ranges therebetween, etc.) of tocilizumab is administered 24 hours (e.g., plus or minus 1, 2, 4, 6, or 8 hours) after the first dose. In some embodiments, a second dose (e.g., of less than 400 mg (e.g., 50 mg, 80 mg, 200 mg, ranges therebetween, etc.) of tocilizumab is administered 36 hours (e.g., plus or minus 1, 2, 4, 6, or 8 hours) after the first dose. In some embodiments, a second dose (e.g., of less than 400 mg (e.g., 50 mg, 80 mg, 200 mg, ranges therebetween, etc.) of tocilizumab is administered 48 hours (e.g., plus or minus 1, 2, 4, 6, or 8 hours) after the first dose. In some embodiments, a second dose (e.g., of less than 400 mg (e.g., 50 mg, 80 mg, 200 mg, ranges therebetween, etc.) of tocilizumab is administered 72 hours (e.g., plus or minus 1, 2, 4, 6, or 8 hours) after the first dose. In some embodiments, a subsequent dose (e.g., after a first, second, third, etc.) is administered with a 4 hours, 8 hours, 12 hours, 24 hours, 2 days, 3 days, 4 days, 5 days, 6 days, 1 week, 2 weeks, 3 weeks, 4 weeks, etc. between administrations.

In some embodiments, methods are provided for the treatment or prevention of viral infection by the co-administration of tocilizumab with one or more additional therapeutics or therapies (e.g., for the treatment of viral infection (e.g., SARS-CoV-2), for treatment of COVID-19, for treatment/prevention of CRS, etc.).

In some embodiments, tocilizumab is co-administered with a suitable antiviral agent. Exemplary, although non-limiting, antiviral agents may be selected from abacavir, acyclovir, adefovir, amantadine, ampligen, amprenavir, arbidol, atazanavir, atripla, balavir, baloxavir marboxil, biktarvy, boceprevir, cidofovir, cobicistat, combivir, daclatasvir, darunavir, delavirdine, descovy, didanosine, docosanol, dolutegravir, doravirine, ecoliever, edoxudine, efavirenz, elvitegravir, emtricitabine, enfuvirtide, entecavir, etravirine, famciclovir, favipravir fomivirsen, fosamprenavir, foscarnet, fosfonet, a fusion inhibitor, ganciclovir, ibacitabine, ibalizumab, idoxuridine, imiquimod, imunovir, indinavir, inosine, an integrase inhibitor, interferon type i, interferon type ii, interferon type iii, interferon, lamivudine, letermovir, lopinavir, loviride, maraviroc, methisazone, moroxydine, nelfinavir, nevirapine, nexavir, nitazoxanide, norvir, nucleoside analogues, oseltamivir, peginterferon alfa-2a, peginterferon alfa-2b, penciclovir, peramivir, pleconaril, podophyllotoxin, a protease inhibitor, pyramidine, raltegravir, remdesivir, a reverse transcriptase inhibitor, ribavirin, rilpivirine, rimantadine, ritonavir, saquinavir, simeprevir, sofosbuvir, stavudine, a synergistic enhancer, telaprevir, telbivudine, tenofovir alafenamide, tenofovir disoproxil, tenofovir, tipranavir, trifluridine, trizivir, tromantadine, truvada, valaciclovir, valganciclovir, vicriviroc, vidarabine, viramidine, zalcitabine, zanamivir (relenza), zidovudine, etc.

In some embodiments, tocilizumab is co-administered with a combination of lopinavir and ritonavir. In some embodiments, tocilizumab is co-administered with alpha-interferon. In some embodiments, tocilizumab is co-administered with an oral combination of lopinavir and ritonavir and nebulized alpha-interferon.

In some embodiments, tocilizumab is co-administered with umifenovir.

In some embodiments, tocilizumab is co-administered with hydroxychloroquine.

In some embodiments, tocilizumab is co-administered with remdesivir.

In some embodiments, tocilizumab is co-administered with glucocorticoids (e.g., high doses of glucocorticoids).

In some embodiments, tocilizumab is co-administered with one or more suitable immunosuppressant drugs. Exemplary, although non-limiting, immunosuppressant drugs may be selected from corticosteroids (e.g., prednisone, budesonide, prednisolone, etc.), Janus kinase inhibitors (e.g., tofacitinib, etc.), calcineurin inhibitors (e.g., cyclosporine, tacrolimus, mTOR inhibitors (e.g., sirolimus, everolimus, etc.), IMDH inhibitors (e.g., azathioprine, leflunomide, mycophenolate, etc.), biologics (e.g., abatacept, adalimumab, anakinra, certolizumab, etanercept, golimumab, infliximab, ixekizumab, natalizumab, rituximab, secukinumab, tocilizumab, ustekinumab, vedolizumab, etc.), monoclonal antibodies (e.g., basiliximab, daclizumab), etc.), etc.

In some embodiments, tocilizumab is co-administered with one or more additional IL-6 antagonists, such as anakinra, sarilumab, ALX-0061, sirukumab, MEDI5117, clazakizumab, and olokizumab.

Although some embodiments herein are directed to the use of tocilizumab to inhibit IL-6 and treat/prevent cytokine release syndrome, the use of other IL-6/IL6Rantagonists, such as anakinra, sarilumab, ALX-0061, sirukumab, MEDI5117, clazakizumab, and olokizumab within the embodiments described herein as being directed to tocilizumab is within the scope of the invention. In some embodiments, a low dose of an IL-6 and/or IL6R antagonist is administered (e.g., <100 mg of anakinra (e.g., 75 mg, 50 mg, 25 mg 10 mg, or ranges therebetween), <200 mg of sarilumab (e.g., 150 mg, 125 mg, 100 mg, 75 mg, 50 mg, 25 mg 10 mg, or ranges therebetween), (e.g., <50 mg of sirukumab (e.g., 40 mg, 25 mg 10 mg, or ranges therebetween), etc. Additionally, any embodiments described herein as employing tocilizumab may also find use with tocilizumab biosimilars (e.g., BAT 1806 (Bio-Thera Solutions), BOW070 (Epirus Biopharmaceuticals), etc.).

In some embodiments, methods are provided for testing, assessing, quantifying, qualifying, evaluating, etc. one or more signs, symptoms, biomarkers, etc. of SARS-CoV-2 infection, COVID-19, cytokine release syndrome, lung infiltration, etc. Any techniques described herein (e.g., chest x-ray, qPCR, ELISA, etc.) for the detection of biomarkers, symptoms, etc. are within the scope herein.

In some embodiments, methods herein comprise testing a subject for the presence of one or more symptoms and/or biomarkers of SARS-CoV-2 infection, COVID-19, CRS, hyperinflammation (e.g., respiratory, pulmonary, etc.), etc. and then administering a dose of tocilizumab (or co-administering) based on the results of the testing.

In some embodiments, methods herein comprise administering a dose of tocilizumab and subsequently testing a subject for the presence of one or more symptoms and/or biomarkers of SARS-CoV-2 infection, COVID-19, CRS, hyperinflammation (e.g., respiratory, pulmonary, etc.), etc. and to determine the success of the treatment.

In some embodiments, provided herein are multiple rounds of testing and treating (e.g., test-treat-test, treat-test-treat, etc.).

Some embodiments herein comprise a step of identifying a subject as being infected with SARS-CoV-2 using a suitable assay or kit. In particular embodiments, a PCR-based assay is used to test a biological sample (e.g., blood, saliva, etc.) from a subject for SARS-CoV-2 infection.

Some embodiments herein comprise performing one or more tests to assess whether a subject suffers from COVID-19. In some embodiments, one or more tests is performed to determine if a subject is critically ill with COVID-19, not critically ill but at high risk for disease advancement, not critically ill and at low risk for disease advancement, suffering from mild COVID-19, suffering from moderate COVID-19, suffering from severe COVID-19, experiencing aberrant respiratory (lung) inflammation, suffering from a cytokine storm, etc. In some embodiments, a treatment course of action (e.g., comprising administering tocilizumab) is selected based on testing using the assays, biomarkers, symptoms, etc. described herein. In some embodiments, the efficacy/success of a treatment course of action (e.g., comprising administering tocilizumab) is measured/assessed based on testing using the assays, biomarkers, symptoms, etc. described herein. Any assays, biomarkers, or symptoms described herein (including in the EXPERIMENTAL section) may be used in embodiments herein to determine a treatment course of action (e.g., whether to administer tocilizumab, dose/regimen of tocilizumab, what to co-administer with tocilizumab, etc.) and/or assess the success of a treatment (e.g., whether to continue/discontinue, whether to alter dose, whether to begin/continue/discontinue a co-therapy, etc.).

In some embodiments, C-reactive protein (CRP) is measured as a useful proxy marker for serum IL-6 levels as well as COVID-19 disease-related inflammation in hospitalized patients, both critically ill and non-critically ill (Refs. 8-10; incorporated by reference in its entirety). Experiments conducted during development of embodiments herein demonstrate that admission CRP is elevated in approximately 90% of hospitalized patients with COVID-19 disease (using an institutional normal range of <5 μg/mL). Critically ill patients with COVID-19 disease tend to have higher CRP than non-critically ill patients (mean, 91 vs 66 μg/mL). Approximately 50% of hospitalized, non-critically ill patients with COVID-19 have CRP of <40 μg/mL, 30% of patients have CRP between 41 and 75 μg/mL, and 20% have CRP above 75 μg/mL.

### EXPERIMENTAL

### Example 1

### Clinical Study of Low-Dose Tocilizumab Administration to Hospitalized, Non-Mechanically-Ventilated Adult Patients with COVID-19 Pneumonitis

Experiments were conducted during development of embodiments herein to study the efficacy of low-dose tocilizumab in treating non-intubated hospitalized adult patients with COVID-19, radiographic pulmonary infiltrate, fever, and C-reactive protein (CRP)>40 mg/L. Experiments were conducted to demonstrate that doses significantly lower than the standards of 400 mg or 8 mg/kg would resolve clinical and laboratory indicators of hyperinflammation. A dose range from 40 to 200 mg was evaluated, with allowance for one repeat dose at 24 to 48 hours. The primary objective was to assess the relationship of dose to fever resolution and CRP response. Thirty-two patients received low-dose tocilizumab, with the majority experiencing fever resolution (75%) and CRP decline consistent with IL-6 pathway abrogation (86%) in the 24-48 hours following drug administration. There was no evidence of a relationship between dose and fever resolution or CRP decline over the dose range of 40-200 mg. Within the 28-day follow-up, 5 (16%) patients died. For patients who recovered, median time to clinical recovery was 3 days (IQR, 2-5). Clinically presumed and/or cultured bacterial superinfections were reported in 5 (16%) patients. Low-dose tocilizumab was associated with rapid improvement in clinical and laboratory measures of hyperinflammation in hospitalized patients with COVID-19.

### Methods

### Study Participants

Eligible patients were those that were hospitalized, aged 18 years or older, and who had: (1) positive polymerase chain reaction test for SARS-CoV-2 RNA, (2) chest radiograph findings such as bilateral ground glass opacities or hazy bilateral infiltrates consistent with viral or atypical pneumonia, (3) documented fever, defined as temperature 38.0° C. in the 24 hours prior to the time of tocilizumab administration as measured by commonly accepted clinical methods (predominantly oral or axillary), and (4)C-reactive protein (CRP) 40 mg/L. Key exclusion criteria included use of invasive mechanical ventilation, scheduled anti-pyretic medications (PRN administration of anti-pyretic following documented fever was allowed), vasopressor medications, active therapy with biologic immunosuppressive or Janus kinase inhibitor medications, and previous receipt of an investigational antiviral agent or off-protocol anti-IL6R therapy.

### Trial Procedures

Enrolled patients were subdivided into two groups (Group A and Group B) based on laboratory signs of hyperinflammation and the presence or absence of risk factors for COVID-19-related mortality (based on extant univariate regressions at the time of study and as agreed to by multidisciplinary panel of institutional experts). Risk factors, nearly all of which were subsequently validated, included: any previous intensive care unit admission; previous non-elective intubation; hospitalization for exacerbation of congestive heart failure or chronic obstructive pulmonary disease in the past 12 months; coronary artery disease requiring percutaneous coronary intervention or coronary artery bypass grafting; stroke with residual neurologic deficit; pulmonary hypertension; home supplemental oxygen use; interstitial lung disease; asthma requiring inhaled corticosteroid use; history of pneumonectomy, lobectomy, or radiation therapy to lung; HIV/AIDS; cancer diagnosis (any stage) receiving non-hormonal treatment; immunodeficiency; end-stage renal disease requiring hemodialysis or peritoneal dialysis; body-mass index ≥30 kg/m2; and inpatient supplemental oxygen requirement >6 L/min at the time of enrollment. Group A patients had CRP ≥75 mg/L and at least one risk factor for mortality. Group B patients had CRP of 40-74 mg/L or lacked risk factors for COVID-19-related mortality. This strategy was utilized to justify testing of a lower dose of tocilizumab in patients deemed at lower risk of death from COVID-19.

After group assignment, patients were treated according to the low-dose tocilizumab algorithm, integrated into usual clinical care FIG. 4). Group A patients received tocilizumab 200 mg or, later, 120 mg. Group B patients received 80 mg or, later, 40 mg. The study was an adaptive design, and all dosage levels were protocol-based and determined on a cohort basis. The study began with 200 mg and 80 mg dose levels. After observing clinical and biochemical responses to the 200 mg and 80 mg dose levels and no safety events, the protocol was transitioned to 120 mg and 40 mg dose levels. Vital signs and laboratory studies were monitored per usual clinical care. CRP was evaluated immediately prior to tocilizumab administration (baseline) and approximately 24 hours following tocilizumab administration.

Patients were eligible for re-dosing with tocilizumab. In the originally developed re-dosing schema, the decision was guided strictly by CRP response. In an amended version of the protocol, however, the re-dosing decision was guided by biochemical and clinical parameters. Patients were re-dosed with tocilizumab if signs of clinical worsening (as defined by increased supplemental oxygen requirement or worsening fever curve determined by maximum temperature) were accompanied by CRP decline of <25% when compared to baseline at the 24-hour assessment (FIG. 4). According to the algorithm used, treating physicians maintained the option of administering off-protocol tocilizumab (generally 400 mg) as indicated per clinical judgment.

Patients were followed for the duration of their hospitalization and were contacted 28 days after tocilizumab administration. At the 28-day timepoint, survival, clinical status (including admission to a medical or assisted living facility), new or persistent supplemental oxygen requirement, and any notable diagnoses or treatments for secondary infection were documented.

### Outcomes

The primary clinical outcome was resolution of fever in the 24-hour period following tocilizumab, defined as a maximum temperature (Tₘₐₓ₂₄ₕᵣₛ)<38·0° C. Fever resolution was chosen for its rapid readout and as a proxy for discharge in high-risk patients with COVID-19 who did not require supplemental oxygen (i.e., in the absence of requirement for supplemental oxygen in a high-risk patient, a fever might prevent discharge while its absence might allow discharge). Patients' requirement for supplemental oxygen was also tracked, so as to inform re-dosing decisions (FIG. 4). The initial primary biochemical outcomes were rate of and time to CRP normalization, guided by earlier tocilizumab-related work. During the conduct of the study, it became apparent that CRP normalization would lag behind a patient's clinical improvement and the patient could be safely discharged before CRP had normalized, making CRP normalization an impractical outcome measure. Therefore the percentage of patients who achieved biochemical response is reported, defined as a CRP reduction ≥25% from baseline in the 24-48 hours after tocilizumab administration, consistent with a decline determined by CRP half-life.

Secondary outcomes included overall survival at 28 days, survival to hospital discharge, rate and duration of non-elective mechanical ventilation, time to mechanical ventilation, rate and duration of vasopressor/inotropic agent utilization, time to vasopressor/inotropic agent utilization, and number of days spent in intensive care unit. One month after enrollment began, duration of supplemental oxygen requirement greater than a patient's baseline was added.

### Post-Hoc Evaluation of Clinical Recovery

Randomized, controlled trials of COVID-19 therapies had not been published at the time of initiation of the study described herein but were published prior to data maturity. To add context to the results, time to clinical recovery was evaluated post-hoc. Using a previously developed seven-point ordinal scale, disease severity was assessed daily, with the patient's worst clinical status and consequent ordinal score recorded for a given day. Recovery was defined as the first day on which a patient achieved a clinical status of either “hospitalization without need for supplemental oxygen or ongoing medical care” or “not hospitalized”. Time to recovery was defined as the time in days from study enrollment to achievement of recovery.

### Statistical Analysis

Descriptive statistics of the clinical and biochemical response rates of patients treated with low-dose tocilizumab were generated. Differences in the clinical and biochemical response rates between different tocilizumab doses were assessed using a two-sided Fisher's exact test, unadjusted for cohort or other risk factors. A p-value of <0.05 was considered to indicate statistical significance. All analyses were performed using Stata (Stata Corp., College Station, Tex., USA). For post hoc analysis, descriptive data summaries are reported as median (interquartile range) or percentages.

### Results

### Trial Population

Twelve patients were assigned to Group A, 8 of whom received 200 mg and 4 of whom received 120 mg. Twenty were assigned to Group B, 15 of whom received 80 mg and 5 of whom received 40 mg FIG. 5). Median time from hospital admission to study enrollment was 1 day (IQR, 1-2 days). All patients were included in the statistical analysis of fever resolution, and 29 patients were included in the statistical analysis of CRP response. Characteristics of the subpopulations are summarized in Table 1.

TABLE 1
Demographic, clinical, and laboratory characteristics of enrolled patients

| | | | | | Overall | |
|---|---|---|---|---|---|---|
| | COVIDOSE | | COVIDOSE | | COVIDOSE | |
| | Group A | | Group B | | Pop. | |
| | n = 12 | | n = 20 | | n = 32 | |
| Age, median (IQR) - years | 70 | (51, 73) | 66 | (41, 73) | 69 | (41, 73) |
| Male sex - n, (%) | 7 | (58.3%) | 9 | (45.0%) | 16 | (50.0%) |
| Race - n, (%) | | | | | | |
| White | 1 | (8.3%) | 0 | (0%) | 1 | (3.1%) |
| Black/African-American | 9 | (75.0%) | 16 | (80.0%) | 25 | (78.1%) |
| Hispanic | 2 | (16.7%) | 1 | (5.0%) | 3 | (9.4%) |
| Multiracial, other, prefer not to identify | 0 | (0.0%) | 3 | (15.0%) | 3 | (9.4%) |
| Comorbidities per patient - n, (%) | | | | | | |
| None | 0 | (0%) | 4 | (20.0%) | 4 | (12.5%) |
| 1 | 2 | (16.7%) | 6 | (30.0%) | 8 | (25.0%) |
| 2 | 4 | (33.3%) | 6 | (30.0%) | 10 | (31.3%) |
| 3 or greater | 6 | (50.0%) | 4 | (20.0%) | 10 | (31.3%) |
| Epidemiologic risk factors from risk | | | | | | |
| group stratification per patient - n, (%) | | | | | | |
| None | 1 | (8.3%) | 12 | (60.0%) | 13 | (40.6%) |
| 1 | 5 | (41.7%) | 5 | (25.0%) | 10 | (31.3%) |
| 2 | 5 | (41.7%) | 3 | (15.0%) | 8 | (25.0%) |
| 3 or greater | 1 | (8.3%) | 0 | (0%) | 1 | (3.1%) |
| Medications, at baseline - n, (%) | | | | | | |
| Hydroxychloroquine | 4 | (33.3%) | 6 | (30.0%) | 10 | (31.3%) |
| Azithromycin | 5 | (41.7%) | 8 | (40.0%) | 13 | (40.6%) |
| Lopinavir-Ritonavir | 1 | (8.3%) | 3 | (15.0%) | 4 | (12.5%) |
| Systemic corticosteroid (within 24 H of | 0 | (0%) | 0 | (0%) | 0 | (0%) |
| enrollment) | | | | | | |
| Supplemental O2 Requirement at | | | | | | |
| enrollment (eligibility) - n, (%) | | | | | | |
| None | 2 | (16.7%) | 6 | (30.0%) | 8 | (25.0%) |
| Low-flow oxygen | 9 | (75.0%) | 13 | (65.0%) | 22 | (68.8%) |
| Non-invasive ventilation or high-flow | 1 | (8.3%) | 1 | (5.0%) | 2 | (6.5%) |
| oxygen | | | | | | |
| C-Reactive Protein baseline, mean (SEM) - | 175 | (24) | 138 | (17) | 152 | (14) |
| mg/L | | | | | | |
| D-dimer baseline, mean (SEM) - pg/mL | 1.98 | (0.50) | 2.2 | (0.96) | 2.12 | (0.62) |
| Ferritin baseline, mean (SEM) - pg/mL | 2022 | (458) | 1151 | (308) | 1478 | (264) |
| COVID-GRAM risk prediction score, | 126.8 | (113.5, 151.8) | 125.2 | (102.3, 158.9) | | |
| median (IQR) | | | | | | |

### Clinical Outcomes

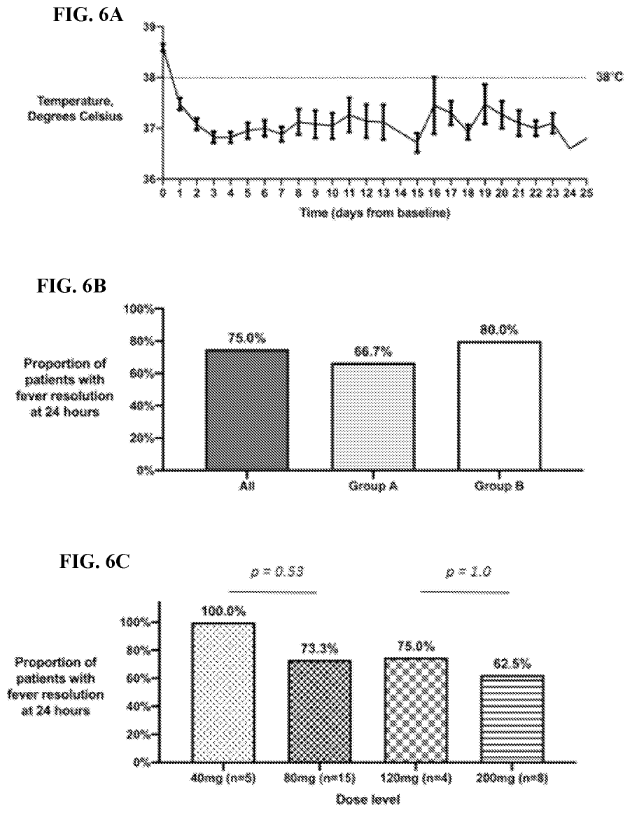

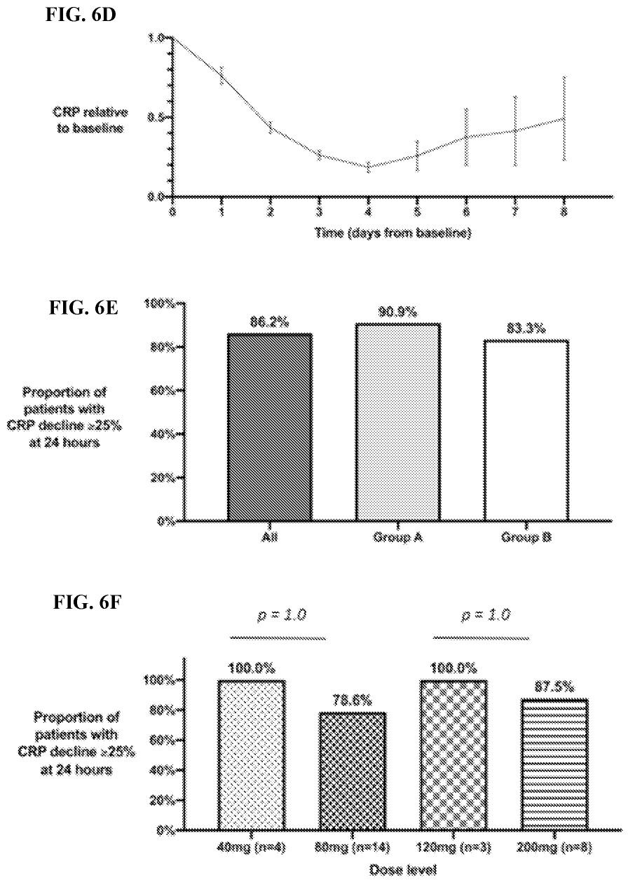

The trend of the maximum daily temperature (mean±standard error of the mean) in patients is shown in FIG. 6A. At 24 hours following tocilizumab administration, 24 patients (75.0%) were afebrile (Tₘₐₓ₂₄ₕᵣₛ<38·0° C.), including 67% of patients in Group A and 80% of those in Group B (FIG. 2B). There was no evidence of a relationship between the tocilizumab dose and rate of fever resolution in the first 24 hours (FIG. 6C). Ten of the 24 patients (42%) with baseline supplemental oxygen requirement had a decrease in maximum oxygen requirement in the first 24 hours following tocilizumab administration.

### Biochemical Outcomes

The CRP trend (mean±standard error of the mean) for the tested population is shown in FIG. 6D. The vast majority (29/32) of patients were evaluable for CRP response, of whom 25 (86%) achieved CRP decrease ≥25%. This included 91% of Group A patients and 83% of Group B patients (FIG. 6E). There was no evidence of a relationship between tocilizumab dose and likelihood of achieving CRP decrease ≥25% (FIG. 6F).

### Post-Hoc Evaluation of Recovery Time and Safety

The 28-day mortality rate was 16% (Table 2). The median time to recovery for the remaining patients was 3 days (IQR, 2-5), and appeared to be associated with the severity of disease at enrollment (Table 2). Culture-proven or clinically suspected ventilator-associated or hospital-acquired pneumonias were identified in 5 (16%) patients.

TABLE 2
Post hoc clinical outcomes in the overall study population and according
to baseline ordinal clinical status and risk group assignment.

| | | Ordinal Score | | | Risk Group | |
|---|---|---|---|---|---|---|
| | | at Baseline | | | Stratification | |
| | | | 4 - Low- | 5 - No | at Baseline | |
| | | 3 - NIV, | flow | supp. | Risk | Risk |
| | | HHFNC | oxygen | oxygen | Group A | Group B |
| | Overall | (n = 4) | (n = 17) | (n = 11) | (n = 12) | (n = 20) |
| Recovery | 27 | 2 | 15 | 10 | 10 | 17 |
| No. of recoveries | | | | | | |
| Median time to | 3 | 11.5 | 4 | 2 | 4.5 | 3 |
| recovery (IQR) - | (2-5) | (10.75- | (3-5) | (2-3) | (2.5- | (2-4) |
| days | | 12.25) | | | 6.75) | |
| Mortality | 5 | 2 | 2 | 1 | 2 | 3 |
| No. of deaths by | | | | | | |
| day 28 | | | | | | |

*Abbreviations: NIV, non-invasive ventilation; HHFNC, heated high-flow nasal cannula.

### CONCLUSIONS

Low-dose tocilizumab was clinically and biochemically active in patients with COVID-19 and related hyperinflammation who did not require invasive ventilation, with no apparent relationship between tocilizumab dose and clinical or biochemical improvement over the studied dose range of 40 to 200 mg. Although the minimum effective dose of tocilizumab was not identified in this clinical trial, the study demonstrated that a tocilizumab dose of 40 mg may be sufficient to blunt the clinical and biochemical signs of COVID-19-related hyperinflammation. Referencing clinical pharmacology arguments, the absence of a dose-response relationship indicates that a dose much lower than those utilized in other COVID-19 studies (400 mg) or approved by regulatory bodies for the treatment of CAR-T-related CRS (4-8 mg/kg) is sufficient to blunt IL-6-mediated hyperinflammation. The experiments conducted during development of embodiments herein provide a COVID-19 therapeutic strategy centered on the administration of low-dose tocilizumab, with re-dosing guided by readily assessable clinical and biochemical responses.

Experiments conducted during development of embodiments herein provide evidence of low-dose tocilizumab's clinical benefit. Marked declines in maximum body temperature and CRP were noted in the 24-48 hours following administration of low-dose tocilizumab-responses that mimic those observed in patients with severe and critical COVID-19 who received tocilizumab 400 mg.

### Example 2

### Clinical Trial Comparing Low Doses of Tocilizumab with Standard of Care Only

A an open-label randomized, controlled trial was conducted during development of embodiments herein in which patients are randomized to receive one of the following three treatments: Standard of care treatments alone (at the discretion of the primary provider), tocilizumab 40 mg+standard of care treatments, or tocilizumab 120 mg+standard of care treatments. The randomization of eligible patients is stratified on remdesivir (an antiviral) and dexamethasone (a corticosteroid), both of which are thought to reduce the time to recovery from COVID-19.

Data was evaluated for 28 patients enrolled in the study for at least 28 days and 27 were evaluable for meta-analysis's primary outcome measure (survival at 28 days). 1 patient was lost to follow-up. Of the 27 patients, 8 were randomized to receive standard of care alone, 9 to tocilizumab 40 mg, and 10 to tocilizumab 120 mg. Two patients died, both in the standard of care arm. Zero of the 19 patients randomized to receive tocilizumab had died. The patients who died were both males ≥70 years old, one of whom was receiving corticosteroids at the time of randomization and the other who was not. Additional secondary outcome measures were evaluated, including requirement for invasive mechanical ventilation and requirement for cardiovascular support. Zero of the patients randomized to receive tocilizumab in the clinical trial required either. A total of three non-COVID-19 secondary infections were declared among the 27 patients, 1 in each of substudy A's three arms.

The data from the study are summarized in Table 3.

TABLE 3

| | | | | Total | | Total |
|---|---|---|---|---|---|---|
| | | Total | | events in | | events in |
| | | events in | Total | patients | Total | patients |
| | Total | patients | randomised | randomised | randomised | randomised |
| Mortality | randomised | randomised | to receive | to receive | to receive | to receive |
| at 28 | to receive | to receive | Anti-IL6 | Anti IL-6 | Anti-IL6 | Anti IL-6 |
| days | control | control | (1) | (1) | (2) | (2) |
| Overall (all | 8 | 2 | 9 | 0 | 10 | 0 |
| patients | | | | | | |
| randomised) | | | | | | |

Patient subgroups:

| | | | | | | |
|---|---|---|---|---|---|---|
| Receipt of | | | | | | |
| corticosteroids* | | | | | | |
| and respiratory | | | | | | |
| support** at | | | | | | |
| baseline | | | | | | |
| Corticosteroids | 0 | 0 | 0 | 0 | 0 | 0 |
| AND NOT | | | | | | |
| supplemental O2 | | | | | | |
| therapy | | | | | | |
| Corticosteroids | | | | | | |
| AND supplemental | 1 | 1 | 3 | 0 | 3 | 0 |
| O2 therapy (O2 ≤ | | | | | | |
| 15 l/min) | | | | | | |
| Corticosteroids | | | | | | |
| AND NIV (O2 | 1 | 0 | 0 | 0 | 0 | 0 |
| flow >15 l/min) | | | | | | |
| Corticosteroids | | | | | | |
| AND IMV | 0 | 0 | 0 | 0 | 0 | 0 |
| (including ECMO) | | | | | | |
| No corticosteroids | | | | | | |
| AND NOT | 2 | 0 | 2 | 0 | 4 | 0 |
| supplemental O2 | | | | | | |
| therapy | | | | | | |
| No corticosteroids | | | | | | |
| AND supplemental | 4 | 1 | 4 | 0 | 3 | 0 |
| O2 therapy (O2 ≤ | | | | | | |
| 15 l/min) | | | | | | |
| No corticosteroids | | | | | | |
| AND NIV (O2 | 0 | 0 | 0 | 0 | 0 | 0 |
| flow >15 l/min) | | | | | | |
| No corticosteroids | | | | | | |
| AND IMV | | | | | | |
| (including ECMO) | 0 | 0 | 0 | 0 | 0 | 0 |
| Unknown | 0 | 0 | 0 | 0 | 0 | 0 |
| Acute organ | | | | | | |
| support at | | | | | | |
| baseline | | | | | | |
| No respiratory | 7 | 2 | 9 | 0 | 10 | 0 |
| support or O2 ≤ | | | | | | |
| 15 l/min only AND | | | | | | |
| NOT CVS support | | | | | | |
| No respiratory | | | | | | |
| support or O2 ≤ | | | | | | |
| 15 l/min only AND | | | | | | |
| CVS support | 0 | 0 | 0 | 0 | 0 | 0 |
| NIV (O2 | | | | | | |
| flow >15 l/min) or | | | | | | |
| IMV (including ECMO) | | | | | | |
| AND NOT CVS | 1 | 0 | 0 | 0 | 0 | 0 |
| support | | | | | | |
| NIV (O2 | | | | | | |
| flow >15 l/min) or | | | | | | |
| IMV (including ECMO) | | | | | | |
| AND CVS support | 0 | 0 | 0 | 0 | 0 | 0 |
| Unknown | 0 | 0 | 0 | 0 | 0 | 0 |
| Age | | | | | | |
| <70 years | 6 | 0 | 7 | 0 | 7 | 0 |
| >=70 years | 2 | 2 | 2 | 0 | 3 | 0 |
| Unknown | 0 | 0 | 0 | 0 | 0 | 0 |
| Sex | | | | | | |
| Male | 7 | 2 | 7 | 0 | 5 | 0 |
| Female | 1 | 0 | 2 | 0 | 5 | 0 |
| Unknown/other | 0 | 0 | 0 | 0 | 0 | 0 |
| Race/ethnicity | | | | | | |
| Black/African- | 6 | 1 | 8 | 0 | 9 | 0 |
| American | | | | | | |
| Hispanic/Latino | 0 | 0 | 1 | 0 | 1 | 0 |
| White/Caucasian | 0 | 0 | 0 | 0 | 0 | 0 |
| Unknown/Did not | 2 | 1 | 0 | 0 | 0 | 0 |
| self-identify | | | | | | |
| Severity of | | | | | | |
| systemic | | | | | | |
| inflammation at | | | | | | |
| baseline | | | | | | |
| C-Reactive protein | 0 | 0 | 3 | 0 | 3 | 0 |
| min to <75 μg/mL | | | | | | |
| C-Reactive | 4 | 1 | 5 | 0 | 3 | 0 |
| protein >=75.0 | | | | | | |
| to <150 μg/mL | | | | | | |
| C-Reactive | 4 | 1 | 1 | 0 | 4 | 0 |
| protein >=150 | | | | | | |
| μg/mL | | | | | | |
| Unknown | 0 | 0 | 0 | 0 | 0 | 0 |

*Minimum dose considered as receipt of corticosteroids (mg/day)
Prednisone 30 mg
Dexamethasone 4.5 mg
Hydrocortisone 150 mg
**Respiratory support defined as:
No supplemental oxygen
Supplemental oxygen ≤15 l/min (e.g. by face mask, nasal cannula)
Non-invasive ventilation (>15 L/min, e.g. by face mask, ‘High Flow’ devices (e.g. HFNC), CPAP, BiPAP etc)
Invasive Mechanical ventilation including ECMO
***Anti-IL6 (1) = 40 mg; Anti-IL6 (2) = 120 mg

### REFERENCES

The following references are herein incorporated by reference in their entireties.
1. Petrilli C M, Jones S A, Yang J, et al. Factors associated with hospitalization and critical illness among 4,103 patients with Covid-19 disease in New York City. medRxiv: New York University; 2020:1-25.
2. Mehta P, McAuley D F, Brown M, Sanchez E, Tattersall R S, Manson J J. COVID-19: consider cytokine storm syndromes and immunosuppression. Lancet 2020:1-2.
3. Gritti G, Raimondi F, Ripamonti D, et al. Use of siltuximab in patients with COVID-19 pneumonia requiring ventilatory support. medRxiv: Azienda Socio Sanitario Territoriale Papa Giovanni XXIII Hospital; 2020:1-17.
4. Liu T, Zhang J, Yang Y, et al. The potential role of IL-6 in monitoring coronavirus disease 2019. 2020.
5. Xu X, Han M, Li T, et al. Effective treatment of severe COVID-19 patients with tocilizumab. China XIV: Respiratory and Critical Care Medicine, The First Affiliated Hospital of University of Science and Technology of China (Anhui Provincial Hospital), No 17 Lujjang Road, Hefei 230001, Anhui, People's Republic of China.; 2020:1-12.
6. A study to evaluate the safety and efficacy of tocilizumab in patients with severe COVID-19 pneumonia (COVACTA). NIH National Library of Medicine, 2020. (Accessed Mar. 30, 2020, at https://clinicaltrials.gov/ct2/show/NCT04320615.)
7. FDA Drug Shortages. Food and Drug Administration of the United States., 2020. (Accessed Apr. 14, 2020, at https://www.accessdata.fda.gov/scripts/drugshortages/default.cfm.)
8. Wallace D J, Angus D C, Seymour C W, Barnato A E, Kahn J M. Critical care bed growth in the United States. A comparison of regional and national trends. Am J Respir Crit Care Med 2015; 191:410-6.
9. Genentech initiates phase III clinical trial of Actemra in hospitalized patients with severe COVID-19 pneumonia. 2020. (Accessed Mar. 19, 2020, at https://www.gene.com/media/press-releases/14841/2020-03-18/genentech-initiates-phase-iii-clinical-t.)
10. Ratain M J, Goldstein D A, Lichter A S. Interventional Pharmacoeconomics-A New Discipline for a Cost-Constrained Environment. JAMA Oncol 2019.
11. America FaDAotUSo. Multi-discipline review: Application number BLA 125276 S-114. In: Research CfDEa, ed.: Food and Drug Administration of the United States of America; 2017.
12. Maini R N, Taylor P C, Szechinski J, et al. Double-blind randomized controlled clinical trial of the interleukin-6 receptor antagonist, tocilizumab, in European patients with rheumatoid arthritis who had an incomplete response to methotrexate. Arthritis Rheum 2006; 54:2817-29.
13. Nishimoto N, Terao K, Mima T, Nakahara H, Takagi N, Kakehi T. Mechanisms and pathologic significances in increase in serum interleukin-6 (IL-6) and soluble IL-6 receptor after administration of an anti-IL-6 receptor antibody, tocilizumab, in patients with rheumatoid arthritis and Castleman disease. Blood 2008; 112:3959-64.
14. Zeng C, Cheng J, Li T, et al. Efficacy and toxicity for CD22/CD19 chimeric antigen receptor T-cell therapy in patients with relapsed/refractory aggressive B-cell lymphoma involving the gastrointestinal tract. Cytotherapy 2020; 22:166-71.
15. Swedish Research Study: REACT COVID-19. 2020. (Accessed Apr. 14, 2020, at swedish.org/for-health-professionals/research/react-covid19.)

1. A method of treating a subject infected with severe acute respiratory syndrome coronavirus 2 (SARS-CoV-2) comprising administering an effective dose of 200 mg or less of tocilizumab to the subject.

2. The method of claim 1, wherein the subject suffers from Coronavirus disease 2019 (COVID-19).

3. The method of claim 2, wherein the subject suffers from mild COVID-19.

4. The method of claim 2, wherein the subject suffers from moderate COVID-19.

5. The method of claim 2, wherein the subject suffers from severe COVID-19.

6. The method of claim 1, wherein the subject exhibits symptoms and/or biomarkers of respiratory inflammation.

7. The method of claim 1, wherein the subject suffers from or is exhibiting a systemic inflammatory response.

8. The method of claim 7, wherein the subject suffers from or exhibits systemic hyperinflammation.

9. The method of claim 8, wherein the hyperinflammation manifests as respiratory and/or pulmonary inflammation.

10. The method of claim 1, wherein the subject does not exhibit symptoms and/or biomarkers of respiratory inflammation.

11. The method of claim 1, wherein the subject exhibits cytokine release syndrome.

12. A method of treating a subject suffering from an aberrant inflammatory response arising from a viral infection, the method comprising administering an effective dose of tocilizumab to a subject experiencing symptoms of aberrant inflammation and or displaying biomarkers of aberrant inflammation.

13. The method of claim 12, wherein the viral infection is an upper respiratory infection.

14. The method of claim 12, wherein the viral infection is a coronavirus infection.

15. The method of claim 14, wherein the subject is infected with severe acute respiratory syndrome coronavirus 2 (SARS-CoV-2).

16. The method of claim 15, wherein the subject suffers from Coronavirus disease 2019 (COVID-19).

17. The method of claim 16, wherein the subject suffers from moderate or severe COVID-19.

18. The method of one of claims 1-17, wherein the effective dose comprises less than 100 mg of tocilizumab.

19. The method of claim 18, wherein the effective dose comprises less than 50 mg of tocilizumab.

20. The method of one of claims 1-19, wherein the subject is administered multiple doses comprising less than 400 mg total of tocilizumab.

21. The method of claim 20, wherein the multiple doses total less than 200 mg of tocilizumab.

22. The method of one of claims 1-21, wherein tocilizumab is administered once daily.

23. The method of one of claims 1-21, wherein tocilizumab is administered once every 2-3 days.

24. The method of one of claims 1-23, wherein tocilizumab is administered subcutaneously.

25. The method of one of claims 1-23, wherein tocilizumab is administered intravenously.

26. The method of one of claims 1-25, wherein the tocilizumab is co-administered with one or more additional therapeutics.

27. The method of claim 26, wherein the subject suffers from COVID-19 and the tocilizumab is co-administered with one or more antiviral agents, immunosuppressants, or anti-inflammatory agents.

28. The method of claim 27, wherein the tocilizumab is co-administered with remdesivir.

29. A method comprising:
(a) testing a subject or a biological sample from a subject to determine the appropriateness of tocilizumab administration to treat/prevent cytokine release syndrome as a result of COVID-19;
(b) administering a dose of tocilizumab consistent with the method of one of claims 1-28.

30. The method of claim 29, wherein testing comprises determining the subject's C-reactive protein level.

31. The method of claim 29, wherein testing comprises determining the subject's serum IL-6 level.

32. The method of claim 29, further comprising:
(c) testing a subject or a biological sample from a subject to determine the effectiveness of the tocilizumab administration.

33. The method of claim 32, further comprising:
(d) determining a treatment course of action based on the testing of step (a) and/or step (c).

34. Use of an effective dose of 200 mg or less of tocilizumab for treating a subject infected with severe acute respiratory syndrome coronavirus 2 (SARS-CoV-2).

35. Use of an effective dose of 200 mg or less of tocilizumab in the manufacture of a medicament for use in a method of extreating a subject infected with severe acute respiratory syndrome coronavirus 2 (SARS-CoV-2).

36. A method of treating a subject infected with severe acute respiratory syndrome coronavirus 2 (SARS-CoV-2) comprising administering a low dose of an 11-6 or IL6R antagonist to the subject.
